(19) United States
(12) Patent Application Publication
(10) Pub. No.: US 2013/0090633 A1
(43) Pub. Date: Apr. 11, 2013

(54) OSMOTIC PATCH PUMP

(71) Applicant: University of Southern California, Los Angeles, CA (US)

(72) Inventor: Gerald E. Loeb, South Pasadena, CA (US)

(73) Assignee: UNIVERSITY OF SOUTHERN CALIFORNIA, Los Angeles, CA (US)

(21) Appl. No.: 13/647,132

(22) Filed: Oct. 8, 2012

Related U.S. Application Data
(60) Provisional application No. 61/544,453, filed on Oct. 7, 2011.

(51) Int. Cl. A61K 9/00

(52) U.S. Cl. CPC A61K 9/0004
USPC 604/892.1; 29/888.02

(57) ABSTRACT

An osmotic patch pump may include a dry agent on a die-cut piece of film that may exert an osmotic pressure when dissolved by a fluid. A chamber may contain the dry agent and have a chamber wall made of a semi-permeable membrane that allows fluid to enter the chamber through the membrane, but does not allow dissolved agent to escape from the chamber through the membrane. A sponge may have a surface in contact with an outer surface of the semi-permeable membrane and may be configured to soak up fluid when placed in contact with the sponge. Flow volume and rate may be controlled by user-operated micro valves. The chamber and fluid communication channels may be embossed on a substrate as part of a simple and low cost manufacturing process.

### CROSS-REFERENCE TO RELATED APPLICATION

This application is based upon and claims priority to U.S. provisional patent application 61/544,453, entitled “OSMOTIC PATCH PUMP FOR LYOPHILIZED DRUGS,” filed Oct. 7, 2011 attorney docket number 028080-0691. The entire content of this application is incorporated herein by reference.

### BACKGROUND

### 1. Technical Field

This disclosure relates to medical devices and, in particular, to osmotic patch pumps.

2. Description of Related Art

The fields of cellular, molecular, and genetic engineering are developing a growing number of new drugs and biologicals for treatment of chronic diseases. Many of these agents are polypeptides, proteins, or large, complex molecules that may need to be kept sterile and administered parenterally, rather than orally. Many may also have limited stability in liquid form. Examples of these include peptides such as calcitonin, glucagons and natriuretic factor, monoclonal antibodies for cancer treatment, cytokines for regulation of immune responses, and growth factors and hormones, such as erythropoietin, insulin, and growth hormone.

It can be challenging to store and dispense unit dosages of lyophilized, powdered, or crystallized agents at a low cost. It can also be difficult for patients to self-administer accurately-controlled doses of them.

### SUMMARY

An osmotic patch pump may include a dry agent, chamber, sponge, injector, and injector fluid communication channel. The dry agent may exert an osmotic pressure when dissolved by a fluid. The chamber may contain the dry agent and have a chamber wall made of a semi-permeable membrane that allows fluid to enter the chamber through the membrane, but does not allow dissolved agent to escape from the chamber through the membrane. The sponge may have a surface in contact with an outer surface of the semi-permeable membrane and may be configured to soak up fluid when placed in contact with the sponge. The injector may be configured to inject dissolved agent into or below a patient's skin. The injector fluid communication channel may allow dissolved agent to flow from the chamber to the injector.

The osmotic patch pump may include a die-cut piece of film within the chamber containing the dry agent.

The osmotic patch pump may include a substrate. A portion of the injector fluid communication channel and/or the chamber may be embossed into the substrate.

The osmotic patch pump may include multiple injector fluid communication channels. Each channel may be configured to channel a different portion of the dissolved agent from the chamber to the injector. One or more of the injector fluid communication channels may each have a user-operable valve that may be configured to controllably block the flow of dissolved agent through the injector fluid communication channel when the valve is closed. The injector fluid communication channels and the user-operable valves may collectively cause the rate at which dissolved agent flows from the chamber to the injector to be a function of the number of valves that are open.

A portion of each injector fluid communication channel may be embossed into the substrate.

The osmotic patch pump may include an exhaust port and one or more exhaust fluid communication channels between the chamber and the exhaust port. One or more of the exhaust fluid communication channels may each have a user-operable valve configured to controllably block the flow of dissolved agent through the channel. The exhaust port, exhaust fluid communication channels, and user-operable valves collectively may cause the volume of dissolved agent that flows from the chamber to the injector to be a function of the number of valves that are open.

Each valve may include a membrane invaginated into the channel in a manner that blocks the flow of dissolved agent thorough the channel. A handle may be affixed to the membrane that can be manually pulled on to remove the membrane from the channel, thereby unblocking the channel, without allowing dissolved agent to escape from the channel.

A portion of each exhaust fluid communication channel may be embossed into the substrate.

The osmotic patch pump may include a filter within an injector fluid communication channel that blocks the passage of un-dissolved dry agent and/or impurities in fluid that enters the chamber through the semi-permeable membrane, but not the passage of dissolved agent.

The osmotic patch pump may include a dissolvable plug within an injector fluid communication channel that blocks dissolved agent from flowing through the channel until the plug is dissolved by fluid surrounding the dissolved agent, thereby insuring that no dissolved agent is injected by the injector until a significant portion of dry agent within the chamber has been dissolved.

These, as well as other components, steps, features, objects, benefits, and advantages, will now become clear from a review of the following detailed description of illustrative embodiments, the accompanying drawings, and the claims.

### BRIEF DESCRIPTION OF DRAWINGS

The drawings are of illustrative embodiments. They do not illustrate all embodiments. Other embodiments may be used in addition or instead. Details that may be apparent or unnecessary may be omitted to save space or for more effective illustration. Some embodiments may be practiced with additional components or steps and/or without all of the components or steps that are illustrated. When the same numeral appears in different drawings, it refers to the same or like components or steps.

FIG. 1 illustrates an example of an osmotic patch pump.

FIG. 2 illustrates example of an osmotic patch pump having an exhaust port and multiple fluid communication channels.

FIG. 3 illustrates an example of the valve that may be used for any of the valves discussed herein.

FIGS. 4A and 4B illustrate a cross-section and top view, respectively, of an example of an osmotic patch pump that may be mass-produced using low-cost materials and processes under sterile conditions.

FIGS. 5A and 5B illustrate a cross-section and top view, respectively, of another example of an osmotic patch pump.

### DETAILED DESCRIPTION OF ILLUSTRATIVE EMBODIMENTS

Illustrative embodiments are now described. Other embodiments may be used in addition or instead. Details that may be apparent or unnecessary may be omitted to save space or for a more effective presentation. Some embodiments may be practiced with additional components or steps and/or without all of the components or steps that are described.

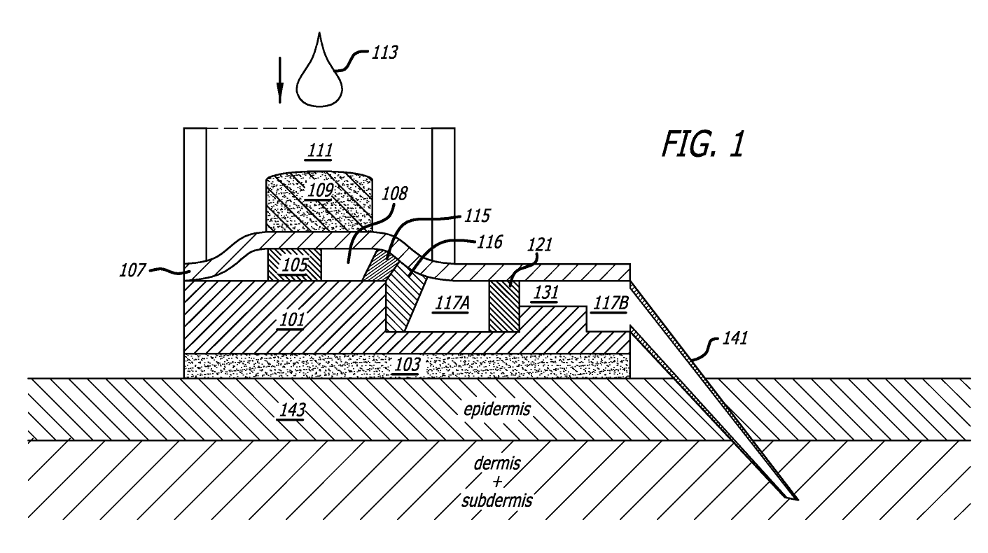

FIG. 1 illustrates an example of an osmotic patch pump. The pump may include a substrate 101, an adhesive layer 103, a dry agent 105, a semi-permeable membrane 107, a chamber 108, a sponge 109, a fluid containment area 111, fluid 113 that may be added before or after the pump is attached to skin of a patient, a dissolvable plug 115, a filter 116, manifolds 117A and 117B, a valve 121, a fluid communication channel 131, and an injector 141 configured to inject fluid flowing into the injector 141 beneath or within the skin 143.

The substrate 101 may be any material. For example, the substrate 101 may be a stiff or semi-rigid polymer that can be embossed to form microfluidic chamber channels and manifolds.

The adhesive layer 103 may be configured to hold the substrate 101 to the skin of a patient for the duration of a treatment and then be readily peeled away. The adhesive layer 103 may include any type of adhesive, such as a biocompatible contact adhesive. A strap may in addition or instead be used to firmly hold the pump against the skin 143 of a patient.

The dry agent 105 may include a controlled amount of a dry chemical substance that is to be administered into or below the skin of a patient's body, such as a polypeptide, protein, or large, complex molecule. Examples of these include peptides such as calcitonin, glucagons and natriuretic factor, monoclonal antibodies for cancer treatment, cytokines for regulation of immune responses, and growth factors and hormones, such as erythropoietin, insulin, and growth hormone. The dry agent may be configured to dissolve when coming in contact with fluid, such as the fluid 113. The dry agent may include a controlled amount of osmotically active salts and any buffers or stabilizers that the chemical substance may require.

Before being placed in the chamber 108, the dry agent 105 may be homogeneously distributed within a large substrate. The large substrate may then be cut by a die into sub-pieces, each having a precise dimension. One of these die-cut sub-pieces may then be placed in the chamber 108. This may allow the amount of dry agent 105 that is within the chamber 108 to be precisely and easily regulated.

The semi-permeable membrane 107 may be a thin layer of a semi-permeable material that permits diffusion of the fluid 113 into the chamber 108, but does not permit dissolved dry agent within the chamber 108 from escaping through the semi-permeable membrane 107. The semi-permeable membrane 107 may also block bacteria or other contaminants that might be in the fluid 113 from passing into the chamber 108. Examples include polyimide and the cellulose or cellophane used in dialysis tubing.

A portion of the chamber 108 may be embossed into the substrate 101. The dry agent 105, including any die-cut substrate containing it, may be within the chamber 108. The semi-permeable membrane 107 may cover the dry agent 105 and may be attached at surrounding locations to the substrate 101, thereby completing the formation of the chamber 108.

The sponge 109 may have a surface that is in physical or very close contact with an outer surface of the semi-permeable membrane 107 that forms a wall of the chamber 108. The sponge 109 may be made of a material that can rapidly absorb and hold a large quantity of fluid relative to its own dry volume. The sponge 109 may cause the fluid 113 that is added to the fluid containment area 111 to stay in contact with the outer surface of the semi-permeable membrane 107 for a long period, notwithstanding movement of the patient while the osmotic patch pump is attached. This may give time for osmosis to cause a significant portion of the fluid 113 to pass through the semi-permeable membrane 107 and into the chamber 108, which may then dissolve the dry agent 105.

The fluid 113 may be of any type that causes the dry agent 105 to dissolve when coming in contact with it. For example, the fluid may be water. The fluid 113 may contain impurities, such as are present in tap water.

The dissolvable plug 115 may be positioned within the manifold 117A so as to block the flow of dissolved agent from the chamber 108 to the injector 141. The dissolvable plug 115 may be made of a material that dissolves when exposed to the fluid in which the agent 105 has been dissolved. The material may be of the type that dissolves slowly in the presence of the fluid, thereby ensuring that no portion of the agent 105 is injected before substantially all of the agent has been dissolved in the chamber 108. For example, the dissolvable plug may be made of a biocompatible solid or gel such as glucose, polyvinyl alcohol, and/or polyethylene glycol. In other configurations, there may not be a dissolvable plug. Although illustrated as separate from the filter 116, the dissolvable plug 115 may instead be contained within the filter 116 and embedded within the interstices of the filter material.

The filter 116 may be positioned within the manifold 117A so as to require all fluid and all dissolved agent to pass through it. The filter 116 may be a porous or filamentous structure that permits the fluid and the dissolved agent to pass through it, but does not permit un-dissolved agent and/or impurities in the fluid 113 to pass through it.

The manifolds 117A and 117B may be configured to provide a confluence for microfluidic flows. The manifolds 117A and 117B may by embossed into the substrate 101. The semi-permeable membrane 107 may cover the embossed area, thereby completing the manifolds 117A and 117B.

The valve 121 may be configured to block or permit microfluidic flow from the manifold 117A into the fluid communication channel 131. The valve 121 may be configured to be easily operated by the patient that is wearing the osmotic patch pump. Examples of the valve 121 are discussed below. There may be several instances of the valve 121, as also discussed below.

The fluid communication channel 131 may be a microfluidic communication channel and may be embossed into the substrate 101. The other portion of the fluid communication channel 131, as well as the other portion of the manifolds 117A and 117B, may be formed by another portion of the semi-permeable membrane 107 or by another type of covering. The fluid communication channel 131 may be sized both in terms of its length and cross-sectional area so as to present a calibrated impedance to microfluidic flow, thereby permitting the rate of this flow to be regulated by these parameters. The fluid communication channel 131 may, in fact, be multiple channels, as discussed in more detail below.

The injector 141 may be affixed to the substrate 101 and/or the semi-permeable membrane 107 and may include a sharp point that easily penetrates the patient's skin 143 when the osmotic patch pump is affixed to the skin 143. The injector 141 may include an internal lumen configured to transport dissolved agent from the manifold 117B into the patient. The injector 141 may have a length that causes its pointed end to rest within or beneath the patient's skin 143 after the osmotic patch pump is affixed to the skin 143 of the patient. The injector 141 may be made of any material, such as stainless steal. The injector 141 may be an intradermal microneedle.

Although illustrated as a tubular structure, the injector 141 could instead be a structure that utilizes surface tension and capillary flow along any hydrophilic surface of a suitably shaped structure that penetrates the epidermis. Such an optional structure for the injector 141 may simplify its attachment to the outlet of the manifold 117B.

At time of use, the osmotic patch pump may be attached to the surface of the skin 143 by the adhesive layer 103 on the bottom surface of substrate 101, taking care to insure that the injector 141 penetrates the skin to a desired depth. The fluid 113 may be applied to the sponge 109 through an opening in the fluid containment area 111. The fluid 113 may diffuse through semi-permeable membrane 107 and come in contact with the dry agent 105. The dry agent 105 may dissolve into the fluid 113. In turn, this may cause the chamber 108 to become hydrostatically pressurized so as to counteract the osmotic pressure associated with the agent 105. The actual pressure may be controlled by the osmolality and solubility of the chemical components of the dry agent 105, the geometry and elasticity of the semi-permeable membrane 107 and the substrate 101, and the rate of egress of fluid through any outlets from the chamber 108, such as the manifold 117A. The rate of flow of the dissolved agent 105 through the channel 131 may be controlled by the microfluidic impedance of the channel 131. The inlet or outlet of the channel 131 may be blocked by the valve 121.

The sequence can thus be summarized as follows: fluid 113 is applied to the sponge 109, the fluid in the sponge 109 is drawn into the chamber 108 by osmosis, the fluid in the chamber 108 dissolves the dry agent 105, and the dissolved dry agent 105 creates osmotic pressure within the chamber 108. The pressurized fluid dissolves the plug 115 and, thereafter, is filtered by the filter 116, passes through the manifold 117A, passes through the fluid communication channel 131, passes through the manifold 117B, and finally passes through the injector 141 into or beneath the skin 143.

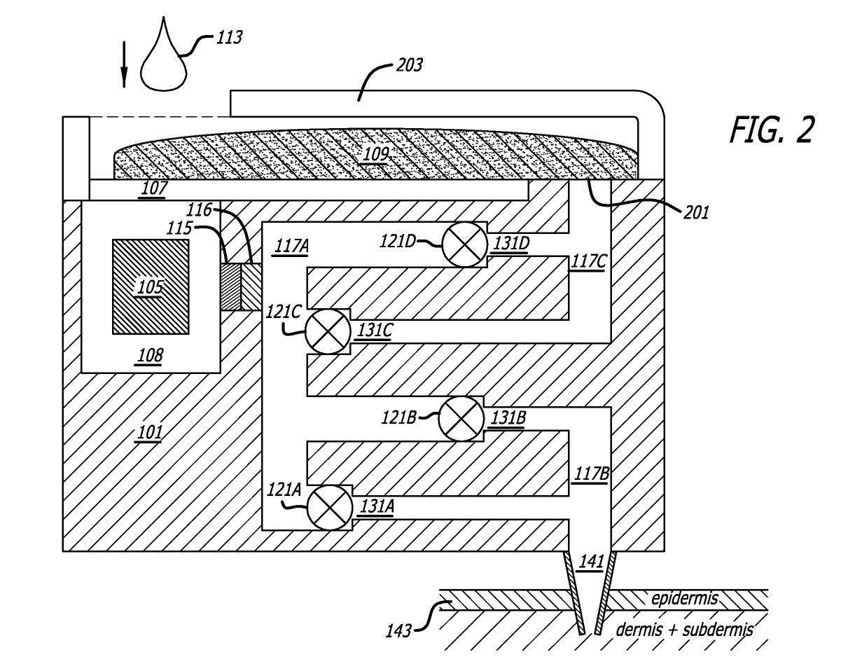

FIG. 2 illustrates another example of an osmotic patch pump having an exhaust port 201 and multiple fluid communication channels. The components in FIG. 2 with the same number as in FIG. 1 may be of the same type, may perform the same functions, and may have the same variations as described above in connection with FIG. 1, except for those types, functions, and variations that are inconsistent.

As illustrated in FIG. 2, a waterproof sheath 203 may be integrated into the osmotic path pump and may form a pocket with the fluid containment area 111. The sheath 203 and the pocket it creates may help keep fluid that has been externally applied to the sponge 109 from escaping while it is being absorbed by the sponge 109 and passes into the chamber 108. The sheath 203 in addition or instead may protect the sponge 109 and the fluid containment area 111 from contamination. Although the pocket formed thereby is illustrated with a large opening for the fluid 113, the opening may be much smaller and protected by a cover flap or a seal. [0043]As illustrated in FIG. 2, there may be multiple injector fluid communication channels, such as injector fluid communication channels 131A and 131B, each controlled by a valve 121A and 121B, respectively. The rate of flow of the dissolved agent from the chamber 108 through the injector 141 into the patient may thus be regulated by opening only one or two of the valves 121A and 121B. The rate of flow may thus depend upon the total number of valves that are opened. A single injector fluid communication channel or more than two injector fluid communication channels, each with an associated valve, may be used instead.

As also illustrated in FIG. 2, there may be an exhaust port 201 that allows some of the dissolved agent to escape and thus not to be delivered into the patient. In this example, the exhaust port 201 allows some of the dissolved agent to escape into the sponge 109. In other configurations, the exhaust port 201 may allow some of the dissolved agent to escape to a different location.

There may similarly be multiple exhaust fluid communication channels, such as exhaust fluid communication channels 131 C and 131 D. Each of these channels may similarly be controlled by a valve, such as the valves 121C and 121D. The volume of flow of the dissolved agent from the chamber through the injector 141 into the patient may thus be regulated by the number of the valves 121C and 121 D that are opened. A single exhaust fluid communication channel or more than two exhaust fluid communication channels, each with an associated valve, may be used instead.

As also illustrated in FIG. 2, the length of the fluid communication channels may vary. For example, one of the fluid communication channels leading to the injector 141 and exhaust port 201 may be short, while the other may be long. The longer channel may present a higher impedance and thus allow less dissolved agent to flow through it during the same period of time, as compared to the shorter channel. This may enable one valve in the set, such as the valve 121 B or the valve 121 D, to coarsely regulate the rate or volume of flow, respectively, while enabling the other valve than the set, such as the valve 121A or 121C to finely regulate the rate of flow or the volume of flow, respectively. There may be similar variations in length and effect when more than two fluid communication channels are used to route the dissolved agent from the chamber 108 to the injector 141 and/or to the exhaust port 201

The sponge 109 may be preloaded with a chemical that would inactivate the agent 105 upon contact. The portion of agent 105 that flows out through exhaust port 201 may be discarded by the patient when the patch pump is removed from the skin, so it may be important to inactivate agent 105 to prevent it from producing undesirable effects on the environment or persons coming into contact with the discarded material. Suitable inactivating chemicals could include acids, alkalis, oxidation agents, enzymes or other chemicals depending on the susceptibilities of agent 105.

The total amount of the agent 105 that flows into the patient recipient thus depends on the amount of the dry agent 105 that is in the chamber 108 and the relative rates of flow through the manifold 117B vs. the manifold 117C. In turn, these characteristics may be controlled by the design of the osmotic patch pump. The design may be modeled and calibrated to facilitate a desired rate and volume.

The channels 131 may be formed by any means, such as by photolithographic etching, additive or subtractive stereo lithography, and/or laser ablation.

Other microfluidic features may be added. For example, one or more check valves may be added to prevent the back flow of fluid from the sponge 109 into the exhaust manifold 117C.

Multiple chambers may be used, each with a different dry agent, all of which may be simultaneously dissolved and pressurized so as to cause their respective dissolved agents to flow into and mix within the manifold 117A. This mixing could be used to catalyze or otherwise enable chemical reactions that would activate, cleave, bond, polymerize, or otherwise modify the separate agents. All of these separate chambers could be next to one another and fed fluid by a common sponge.

Electronic or chemical means may be added to heat the agent 105 as it passes through the channel 131 or the manifold 117, thereby accelerating a desired chemical reaction.

Sensing technology may be incorporated to measure the actual rate or volume of flow through the channel 131 or the manifold 117 so as to monitor the administration of the agent 105. Related control technology may be added, such as one or more controllable valves, to effectuate changes in the monitored rate or volume, based on the output of the sensors.

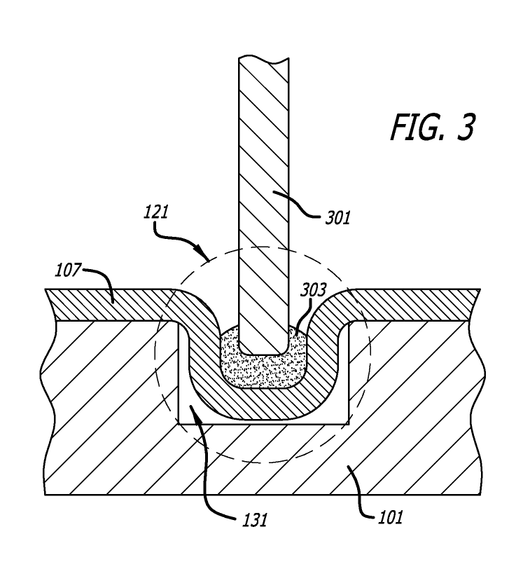

FIG. 3 illustrates an example of the valve 121 that may be used for any of the valves discussed herein. The valve 121 may be operated by the patient or by someone else at the time of administration or at an earlier time. The components in FIG. 3 with the same number as in FIGS. 1 and 2 may be of the same type, may perform the same functions, and may have the same variations as described above in connection with FIGS. 1 and 2, except for those types, functions, and variations that are inconsistent.

The valve 121 may include the semi-permeable membrane 107 invaginated into the channel 131 in a manner that blocks the flow of dissolved agent thorough the channel. The valve 121 may include a short handle 301 that is affixed to the semi-permeable membrane 107 at the location of the invagination by an attachment connection 303, such as an adhesive compound or thermoplastic fusion. The handle 301 may be manually pulled on to remove the semi-permeable from the channel. This may unblock the channel without allowing dissolved agent to escape from the channel. The strength of the attachment connection 303 may be calibrated so that it remains attached to the semi-permeable membrane 107 until the channel is fully opened, but then detaches from the semi-permeable membrane upon continued application of force, thereby preventing the semi-permeable 107 from being ruptured or otherwise damaged. A tab or other protrusion may be used instead of the handle 301.

If the top of channel 131 or manifold 117 is formed from a material other than the semi-permeable membrane 107, the valve may be formed by an invagination of this other material in the same manner as described in the previous paragraph.

One or more of the valves 121 may be of a different design. For example, one or more of the valves 121 may be configured to be operated pneumatically, magnetically, electrolytically or electronically.

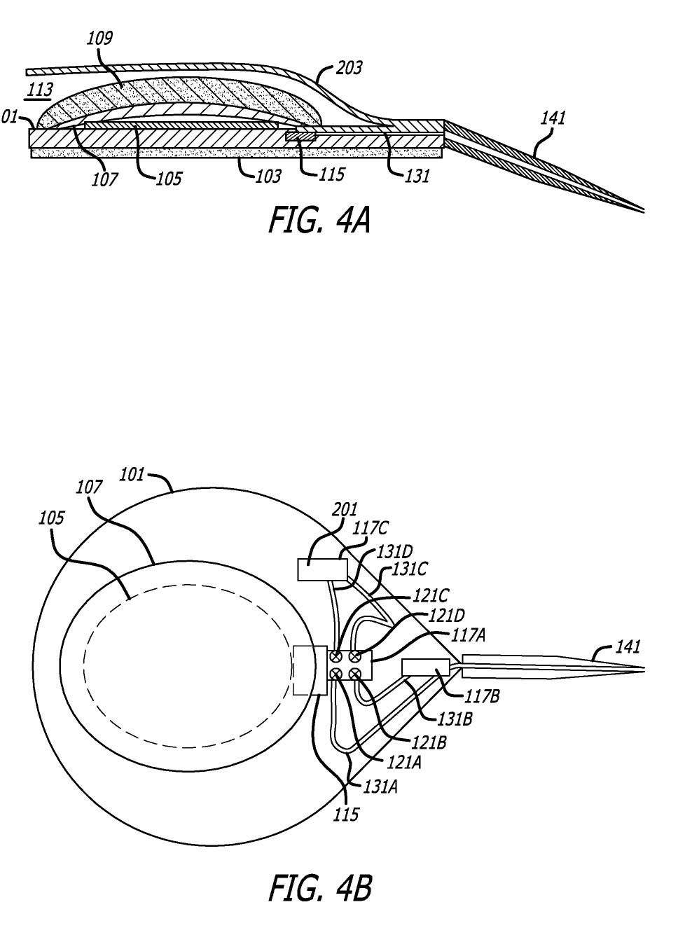

FIGS. 4A and 4B illustrate a cross-section and top view, respectively, of an example of an osmotic patch pump that may be mass-produced using low-cost materials and processes under sterile conditions. The components in FIGS. 4A and 4B with the same number as in FIGS. 1, 2, and 3 may be of the same type, may perform the same functions, and may have the same variations as described above in connection with FIGS. 1, 2, and 3, except for those types, functions, and variations that are inconsistent.

The substrate 101 may be die-cut to the desired shape and embossed with depressions that may form the manifolds 117 and the channels 131. The agent 105 may be lyophilized under sterile conditions to form a solid sheet that is die-cut to provide individual pieces with controlled volume, one of which may be deposited onto the region of substrate 101 where the chamber 108 may eventually be formed. Alternatively, a controlled volume of a solution or gel containing the agent 105 may be deposited onto this region of the substrate 101 and lyophilized or air-dried in place. The filter 116, and the option dissolvable plug 115, may be deposited at the outlet of the chamber 108 into the manifold 117A. The semi-permeable membrane 107 may then be attached to the substrate 101, forming the enclosed space of the chamber 108. The semi-permeable membrane 107 may also form the top cover of the manifolds 117 and the channels 131. Alternatively, these may be formed by attaching part of the sheath 203, as illustrated in FIG. 4A.

The semi-permeable membrane 107 and the sheath 203 may be attached to flush surfaces of the substrate 101 by any means, such as by thermoplastic welding, ultrasonic bonding, or chemical adhesives. In order to incorporate the embodiment of the valve 121 that is illustrated in FIG. 3, the semi-permeable membrane 107 or sheath 107 may be invaginated into corresponding depressions in the substrate 101 by any means, such as by using heat or pressure. A separate instance of the handle 301 may be added via attachment connection 303 to each invagination.

The sponge 109 and any remaining components may be added on top of semi-permeable membrane 107, but avoiding the locations of the handles 301 (not illustrated in FIG. 4).

Injector 141 may be attached to the outlet of the manifold 117B. The adhesive layer 103 may be applied to the bottom of the substrate 101. The completed and loaded osmotic patch pump may then be put into a sterile wrapper to protect the adhesive 103 and the injector 141 (not illustrated). The sterile wrapper may be made from a material with low permeability to moisture to prevent premature activation by absorption of ambient humidity.

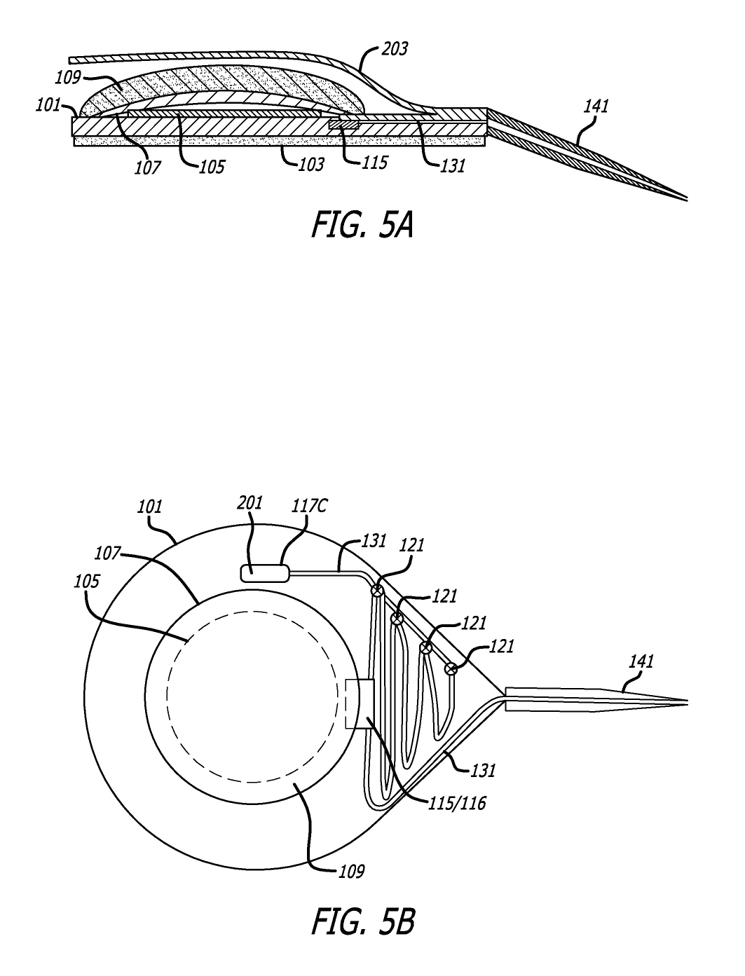

FIGS. 5A and 5B illustrate a cross-section and top view, respectively, of another example of an osmotic patch pump. The components in FIGS. 5A and 5B with the same number as in FIGS. 1, 2, 3, and 4A and 4B may be of the same type, may perform the same functions, and may have the same variations as described above in connection with FIGS. 1, 2, 3, and 4A and 4B, except for those types, functions, and variations that are inconsistent. As illustrated in these figures, the valves 121 only control the flow of dissolved agent to the exhaust port 201, thus regulating the volume that is injected into the patient. No user control is provided for regulating the rate of this flow, except to the extent that the rate may be diminished by exhausting some of the flow. Such a configuration may be useful to enable dispensing the pump to comply with a prescription for a specified amount of agent 105 to be delivered wherein that specified amount is less than the total amount of agent 105 contained in the osmotic patch pump as manufactured.

The osmotic patch pump that has been described thus uses osmotic principles to hydrate and pressurize a drug, biological, or other therapeutic or diagnostic agent, which may be deposited as a thin layer on a stiff substrate and sealed with a semi-permeable membrane covered by a sponge. The agent to be delivered may be deposited as a die-cut piece of a previously dried film, or it could be deposited as a solution or suspension and freeze-dried in place.

When the patient is ready to use the osmotic patch pump, tap water may be applied through the fluid entry zone under the waterproof sheath 203, where it may be soaked up by the sponge 109. Various package and sealing options are possible, including putting the entire device in a disposable envelope or clamshell package, temporarily closing the fluid entry zone by a peelable flap of the sheath attached to the substrate, and/or installing a removable protective sheath over the injector.

Water may pass through the semi-permeable membrane where it may hydrate the agent which may include a drug and buffer salts. The amount of salt may establish an equilibrium point between the osmotic pressure and the hydrostatic pressure that develops in the enclosed space (dry salts such as sodium chloride or potassium chloride or magnesium sulfate may have an osmotic pressure equivalent to ˜200 psi).

The hydrostatic pressure may force the dissolved drug through the filter 116 and the microfluidic flow control channels embossed into the substrate 101. If necessary, the start of delivery of the drug can be delayed by incorporating the dissolvable plug 115 so that essentially all of the dry agent 105 is dissolved and the equilibrium hydrostatic pressure is reached before the dissolved agent starts to flow out of the injector 141.

The injector 141 may enter the skin as the patch is applied and adhered to the skin. A removable or puncturable sheath may be added to protect the sharp end of the injector 141 before insertion into the skin.

There may also be a control channel equipped with one or more plugs (black dots labeled “fused valves”) that can be removed manually by the patient using a rod or pull-tabs. When opened, these valves shunt different portions of the flow to the exhaust port, which may simply be an opening in the microfluidic channel that leads into the sponge outside the semi-permeable membrane. There, the unused drug may mix with the water in the sponge and ma be discarded with the patch when detached from the skin.

The timing, total amount of dissolved agent, and/or the rate of its delivery may be controlled according to some automated measurement, such as heart rate, blood glucose, and/or concentration. To facilitate this, one or more of the manually operated valves that have been discussed may be replaced by microfluidic valves that can be actuated electronically by a controller according to those measured values, a timer, and/or another external signal or criteria.

The resulting osmotic patch pump may thus be single-use, disposable, and low-cost. It may provide an adjustable and accurate dosage and infusion rate to an intra- or subdermal injection site. The agent may be stored in a dry, solid, and sterile form. Hydration and filtering at time of administration may be automatic.

The components, steps, features, objects, benefits, and advantages that have been discussed are merely illustrative. None of them, nor the discussions relating to them, are intended to limit the scope of protection in any way. Numerous other embodiments are also contemplated. These include embodiments that have fewer, additional, and/or different components, steps, features, objects, benefits, and advantages. These also include embodiments in which the components and/or steps are arranged and/or ordered differently.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

All articles, patents, patent applications, and other publications that have been cited in this disclosure are incorporated herein by reference.

The phrase “means for” when used in a claim is intended to and should be interpreted to embrace the corresponding structures and materials that have been described and their equivalents. Similarly, the phrase “step for” when used in a claim is intended to and should be interpreted to embrace the corresponding acts that have been described and their equivalents. The absence of these phrases from a claim means that the claim is not intended to and should not be interpreted to be limited to these corresponding structures, materials, or acts, or to their equivalents.

The scope of protection is limited solely by the claims that now follow. That scope is intended and should be interpreted to be as broad as is consistent with the ordinary meaning of the language that is used in the claims when interpreted in light of this specification and the prosecution history that follows, except where specific meanings have been set forth, and to encompass all structural and functional equivalents.

Relational terms such as “first” and “second” and the like may be used solely to distinguish one entity or action from another, without necessarily requiring or implying any actual relationship or order between them. The terms “comprises,” “comprising,” and any other variation thereof when used in connection with a list of elements in the specification or claims are intended to indicate that the list is not exclusive and that other elements may be included. Similarly, an element preceded by an “a” or an “an” does not, without further constraints, preclude the existence of additional elements of the identical type.

None of the claims are intended to embrace subject matter that fails to satisfy the requirement of Sections 101, 102, or 103 of the Patent Act, nor should they be interpreted in such a way. Any unintended coverage of such subject matter is hereby disclaimed. Except as just stated in this paragraph, nothing that has been stated or illustrated is intended or should be interpreted to cause a dedication of any component, step, feature, object, benefit, advantage, or equivalent to the public, regardless of whether it is or is not recited in the claims.

The abstract is provided to help the reader quickly ascertain the nature of the technical disclosure. It is submitted with the understanding that it will not be used to interpret or limit the scope or meaning of the claims. In addition, various features in the foregoing detailed description are grouped together in various embodiments to streamline the disclosure. This method of disclosure should not be interpreted as requiring claimed embodiments to require more features than are expressly recited in each claim. Rather, as the following claims reflect, inventive subject matter lies in less than all features of a single disclosed embodiment. Thus, the following claims are hereby incorporated into the detailed description, with each claim standing on its own as separately claimed subject matter.

The invention claimed is:

1. An osmotic patch pump comprising:
a dry agent that exerts an osmotic pressure when dissolved by a fluid;
a chamber containing the dry agent and having a chamber wall made of a semi-permeable membrane that allows fluid to enter the chamber through the membrane, but that does not allow dissolved agent to escape from the chamber through the membrane;
a sponge having a surface in contact with an outer surface of the semi-permeable membrane and configured to soak up fluid when placed in contact with the sponge;
an injector configured to inject dissolved agent into or below a patient's skin; and
an injector fluid communication channel that allows dissolved agent to flow from the chamber to the injector.

2. The osmotic patch pump of claim 1 further comprising a die-cut piece of film within the chamber containing the dry agent.

3. The osmotic patch pump of claim 1 further comprising a substrate and wherein a portion of the chamber is embossed into the substrate.

4. The osmotic patch pump of claim 3 wherein at least a portion of the injector fluid communication channel is embossed into the substrate.

5. The osmotic patch pump of claim 1 further comprising:
multiple injector fluid communication channels, each configured to channel a different portion of the dissolved agent from the chamber to the injector; and
a user-operable valve configured to controllably block the flow of dissolved agent through one of the injector fluid communication channels when the valve is closed;
wherein the one of the injector fluid communication channels and the user-operable valve collectively cause the rate at which dissolved agent flows from the chamber to the injector to be greater when the valve is open and less when the valve is closed.

6. The osmotic patch pump of claim 5 further comprising:
for each of the injector fluid communication channels, a user-operable valve configured to controllably block the flow of dissolved agent through the injector fluid communication channel when the valve is closed;
wherein the multiple injector fluid communication channels and the user-operable valves collectively cause the rate at which dissolved agent flows from the chamber to the injector to be a function of the number of valves that are open.

7. The osmotic patch pump of claim 5 wherein the valve includes a membrane invaginated into one of the channels in a manner that blocks the flow of dissolved agent thorough the channel and an associated handle that is affixed to the membrane that can be manually pulled on to remove the membrane from the channel, thereby unblocking the channel, but without allowing dissolved agent to escape from the channel.

8. The osmotic patch pump of claim 5 further comprising a substrate and wherein at least a portion of each injector fluid communication channel is embossed into the substrate.

9. The osmotic patch pump of claim 1 further comprising:
an exhaust port;
an exhaust fluid communication channel between the chamber and the exhaust port; and
a user-operable valve configured to controllably block the flow of dissolved agent through the exhaust fluid communication channel,
wherein the exhaust port, exhaust fluid communication channel, and user-operable valve collectively cause the volume of dissolved agent that flows from the chamber to the injector to be greater when the valve is closed and less when the valve is open.

10. The osmotic patch pump of claim 9 further comprising:
multiple exhaust fluid communication channels, each configured to channel a different portion of dissolved agent from the chamber to the exhaust port; and
for each of the exhaust fluid communication channels, a user-operable valve configured to controllably block the flow of dissolved agent through the exhaust fluid communication channel,
wherein the exhaust port, exhaust fluid communication channels, and user-operable valves collectively cause the volume of dissolved agent that flows from the chamber to the injector to be a function of the number of valves that are open.

11. The osmotic patch pump of claim 9 wherein the valve includes a membrane invaginated into one of the exhaust channels in a manner that blocks the flow of dissolved agent thorough the channel and an associated handle that is affixed to the membrane that can be manually pulled on to remove the membrane from the channel, thereby unblocking the channel without allowing dissolved agent to escape from the channel.

12. The osmotic patch pump of claim 9 further comprising a substrate and wherein at least a portion of the exhaust fluid communication channel is embossed into the substrate.

13. The osmotic patch pump of claim 12 further comprising a substrate and wherein a portion of the chamber, the injector fluid communication channel, and the exhaust fluid communication channel are embossed into the substrate.

14. The osmotic patch pump of claim 9 further comprising:
multiple injector fluid communication channels, each configured to channel a different portion of dissolved agent from the chamber to the injector; and
for each injector fluid communication channel, a user-operable valve configured to controllably block the flow of dissolved agent through the injector fluid communication channel when the valve is closed;
wherein the injector fluid communication channels and the user-operable valves collectively cause the rate at which dissolved agent flows from the chamber to the injector to be a function of the number of valves that are open.

15. The osmotic patch pump of claim 1 further comprising a filter within the injector fluid communication channel that blocks the passage of un-dissolved agent or impurities in fluid that enters the chamber through the semi-permeable membrane, but not the passage of dissolved agent.

16. The osmotic patch pump of claim 1 further comprising a dissolvable plug within the injector fluid communication channel that blocks dissolved agent from flowing through the channel until the plug is dissolved by fluid surrounding the dissolved agent, thereby insuring that no dissolved agent is injected by the injector until a significant portion of dry agent within the chamber has been dissolved.

17. An osmotic patch pump comprising:
a dry agent that exerts an osmotic pressure when dissolved by a fluid;
a die-cut piece of film containing the dry agent;
a chamber containing the die-cut piece of film and the dry agent and having a chamber wall made of a semi-permeable membrane that allows fluid to enter the chamber through the membrane, but that does not allow dissolved agent to escape from the chamber through the membrane;
an injector configured to inject dissolved agent into or below a patient's skin; and
an injector fluid communication channel that allows dissolved agent to flow from the chamber to the injector.

18. An osmotic patch pump comprising:
a dry agent that exerts an osmotic pressure when dissolved by a fluid;
a chamber containing the dry agent and having a chamber wall made of a semi-permeable membrane that allows fluid to enter the chamber through the membrane, but that does not allow dissolved agent to escape from the chamber through the membrane;
an injector configured to inject dissolved agent into or below a patient's skin; and
multiple injector fluid communication channels, each configured to channel a different portion of dissolved agent from the chamber to the injector; and
for each injector fluid communication channel, a user-operable valve configured to controllably block the flow of dissolved agent through the injector fluid communication channel when the valve is closed;
wherein the multiple injector fluid communication channels and the user-operable valves collectively cause the rate at which dissolved agent flows from the chamber to the injector to be a function of the number of valves that are open.

19. A method of making an osmotic patch pump comprising:
creating a substrate for the pump;
positioning an injector that is configured to inject dissolved agent into or below a patient's skin on the substrate;
embossing a portion of a chamber and a portion of an injector fluid communication channel from the portion of the chamber to the injector into the substrate;
placing a dry agent that exerts an osmotic pressure when dissolved by a fluid within the portion of the chamber; and
completing the chamber by affixing a semi-permeable membrane to the substrate.

20. The method of claim 19 further comprising embossing an exhaust fluid communication channel from the portion of the chamber to an exhaust port.
